SEQUENCE LISTING (21 sequences)

SEQ ID NO: 1 - protein, 5 aa, Artificial Sequence
  HCDR1, EGFR binding arm
TYGMH

SEQ ID NO: 2 - protein, 17 aa, Artificial Sequence
  HCDR2, EGFR binding arm
VIWDDGSYKYYGDSVKG

SEQ ID NO: 3 - protein, 16 aa, Artificial Sequence
  HCDR3, EGFR binding arm
DGITMVRGVMKDYFDY

SEQ ID NO: 4 - protein, 11 aa, Artificial Sequence
  LCDR1, EGFR binding arm
RASQDISSALV

SEQ ID NO: 5 - protein, 7 aa, Artificial Sequence
  LCDR2, EGFR binding arm
DASSLES

SEQ ID NO: 6 - protein, 9 aa, Artificial Sequence
  LCDR3, EGFR binding arm
QQFNSYPLT

SEQ ID NO: 7 - protein, 5 aa, Artificial Sequence
  HCDR1, c-Met binding arm
SYGIS

SEQ ID NO: 8 - protein, 17 aa, Artificial Sequence
  HCDR2, c-Met binding arm
WISAYNGYTNYAQKLQG

SEQ ID NO: 9 - protein, 10 aa, Artificial Sequence
  HCDR3, c-Met binding arm
DLRGTNYFDY

SEQ ID NO: 10 - protein, 11 aa, Artificial Sequence
  LCDR1, c-Met binding arm
RASQGISNWLA

SEQ ID NO: 11 - protein, 7 aa, Artificial Sequence
  LCDR2, c-Met binding arm
AASSLLS

SEQ ID NO: 12 - protein, 9 aa, Artificial Sequence
  LCDR3, c-Met binding arm
QQANSFPIT

SEQ ID NO: 13 - protein, 125 aa, Artificial Sequence [sequence omitted]

SEQ ID NO: 14 - protein, 107 aa, Artificial Sequence [sequence omitted]

SEQ ID NO: 15 - protein, 119 aa, Artificial Sequence
  VH, c-Met binding arm
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Artificial Sequence
  VL, c-Met binding arm
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Artificial Sequence
  Heavy chain 1
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Artificial Sequence
  Light chain 1
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Artificial Sequence
  Heavy chain 2
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Artificial Sequence
  Light chain 2
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 1210 aa, Homo sapiens [sequence omitted]

(19) United States
(12) Patent Application Publication
(10) Pub. No.: US 2022/0298248 A1
(43) Pub. Date: Sep. 22, 2022

(54) Treatment of Cancers Lacking EGFR- Activating Mutations

(71) Applicant: Janssen Biotech, Inc., Horsham, PA (US)

(72) Inventors: Benjamin Henley, Mount Laurel, NJ (US); Sheri Moores, Wayne, PA (US)

(21) Appl. No.: 17/687,984

(22) Filed: Mar. 7, 2022

Related U.S. Application Data
(60) Provisional application No. 63/158,552, filed on Mar. 9, 2021.

(51) Int. Cl. C07K 16/28; A61P 35/00; A61P 11/00; A61K 45/06; A61K 39/395

(52) U.S. Cl. CPC C07K 16/2863; A61P 35/00; A61P 11/00; A61K 45/06; A61K 39/39558; A61K 2039/505

(57) ABSTRACT

The present invention relates to treatment of subjects having cancers with tumors lacking an at least one EGFR-activating mutation.

### CROSS-REFERENCE TO RELATED APPLICATIONS

This application claims priority to U.S. Provisional Application Ser. No. 63/158,552, filed 9 Mar. 2021. The entire contents of the aforementioned application are incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “JBI6507USNP1SEQLIST.txt”, creation date of Mar. 1, 2022 and having a size of 29 KB. The sequence listing submitted via EFS-Web is part of the specification and is herein incorporated by reference in its entirety.

### FIELD

The present invention relates to treatment of subjects having a cancer with tumors lacking an at least one EGFR-activating mutation.

### BACKGROUND

The individual roles of both epidermal growth factor receptor (EGFR) and receptor tyrosine kinase mesenchymal-epithelial transition factor (c-Met) in cancer is well established, making these targets attractive for combination therapy. Both receptors signal through the same survival and anti-apoptotic pathways (ERK and AKT); thus, inhibiting the pair in combination may limit the potential for compensatory pathway activation thereby improving overall efficacy.

Molecular segmentation of advanced non-small cell lung cancer (NSCLC) based on oncogenic driver mutations has improved the overall survival and quality of life for patients with actionable driver mutations.

Amivantamab is a bispecific antibody that targets EGFR and c-MET. Its clinical activity is being investigated across a range of EGFR-activating mutations in clinical trials, but has not been evaluated for the treatment of lung cancers that are positive for EGFR but lack the EGFR activating mutations.

### SUMMARY

There is a need for improved therapeutics or combination of therapeutics to develop more effective treatment of cancers having tumors comprising EGFR lacking activating mutations.

The disclosure provides a method of treating a subject having a cancer that is positive for EGFR and lacks an at least one EGFR-activating mutation, comprising administering a therapeutically effective amount of an isolated bispecific anti-epidermal growth factor receptor (EGFR)/hepatocyte growth factor receptor (c-Met) antibody to the subject having cancer that is positive for EGFR and lacks an at least one EGFR-activating mutation.

The disclosure also provides a method of treating a subject having a cancer with a bispecific anti-EGFR/c-Met antibody, comprising:

a) providing a biological sample from the subject;

b) determining presence or absence of an EGFR-activating mutation in the sample;

c) administering or providing for administration the bispecific anti-EGFR/c-Met antibody to the subject determined to lack an EGFR-activating mutation.

In one embodiment, the at least one activating mutation is a mutation which increases at least one biological activity of EGFR.

In one embodiment, the at least one biological activity of EGFR is selected from the group consisting of tyrosine kinase activity, ligand-independent signaling, increased cell proliferation, signaling to MAPK/ERK pathways, gene transcription, dimerization (EGFR:EGFR), and heterodimerization (EGFR:HER2 or EGFR:HER3).

In one embodiment, the at least one activating mutation which increases the at least one biological activity of EGFR comprises at least one mutation selected from the group consisting of L718Q, G719A, G719X (X being any amino acid), L861X (X being any amino acid), L858R, E746K, L747S, E749Q, A750P, A755V, V765M, C797S, L858P or T790M substitution, deletion of E746-A750, deletion of R748-P753, insertion of Ala (A) between M766 and A767, insertion of Ser, Val and Ala (SVA) between S768 and V769, insertion of Asn and Ser (NS) between P772 and H773, insertion of one or more amino acids between D761 and E762, A763 and Y764, Y764 and Y765, M766 and A767, A767 and V768, S768 and V769, V769 and D770, D770 and N771, N771 and P772, P772 and H773, H773 and V774, V774 and C775, one or more deletions in EGFR exon 20, one or more insertions in EGFR exon 20, S768I, L861Q and G719X (X being any amino acid).

In one embodiment, the method further comprises determining presence or absence of at least one mutation in any one gene selected from the group consisting of KRAS, PIK3CA, and PTEN, and administering or providing for administration the bispecific anti-EGFR/c-Met antibody to the subject determined to have the EGFR lacking activating mutations and determined to lack at least one mutation in any one gene selected from the group consisting of KRAS, PIK3CA, and PTEN.

In one embodiment, the at least one mutation in KRAS is selected from the group consisting of G12V, G12C, G12A and G12D.

In one embodiment, the at least one mutation in KRAS is G12C.

In one embodiment, the at least one mutation in PI3K is selected from the group consisting of E545K, H1047L, and PI3K amplification.

In one embodiment, the at least one mutation in PTEN is PTEN deletion.

In one embodiment, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In one embodiment, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

In one embodiment, the bispecific anti-EGFR/c-Met antibody is an IgG1 isotype.

In one embodiment, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In one embodiment, the bispecific anti-EGFR/c-Met antibody comprises one or more Fc silencing mutations.

In one embodiment, the one or more Fc silencing mutations decrease affinity to Fcγ receptors.

In one embodiment, the one or more Fc silencing mutations comprise V234A/G237A/P2385/H268A/V309L/A330S/P331S.

In one embodiment, the bispecific anti-EGFR/c-Met antibody comprises a biantennary glycan structure with a fucose content between about 1% to about 15%.

In one embodiment, the subject is relapsed or resistant to treatment with one or more prior anti-cancer therapies.

In one embodiment, the one or more prior anti-cancer therapies comprises one or more chemotherapeutic agents, checkpoint inhibitors, targeted anti-cancer therapies or kinase inhibitors, or any combination thereof.

In one embodiment, the one or more prior anti-cancer therapies comprises carboplatin, paclitaxel, gemcitabine, cisplatin, vinorelbine, docetaxel, palbociclib, crizotinib, PD-(L)1 axis inhibitor, an inhibitor of EGFR, an inhibitor of c-Met, an inhibitor of HER2, an inhibitor of HER3, an inhibitor of HERO, an inhibitor of VEGFR, an inhibitor of AXL, erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib, or any combination thereof.

In one embodiment, the subject is treatment naïve.

In one embodiment, cancer that is positive for the EGFR lacking activating mutations is positive for at least one mutation in a gene selected from the group consisting of ALK, APC, BRAF, BRCA1, BRCA2, CDKN2A, CDKN2B, CTNNB1, ERBB2, ERBB3, FGFR3, KIT, LRP1B, MET, MLH1, MSH3, NOTCH1, NTRK1, RET, ROS1, STK11, TP53, and VEGFA.

In one embodiment, the cancer is lung cancer, gastric cancer, colorectal cancer, brain cancer, cancer derived from epithelial cells, breast cancer, ovarian cancer, colorectal cancer, anal cancer, prostate cancer, kidney cancer, bladder cancer, head and neck cancer, pharynx cancer, cancer of the nose, pancreatic cancer, skin cancer, oral cancer, cancer of the tongue, esophageal cancer, vaginal cancer, cervical cancer, cancer of the spleen, testicular cancer, gastric cancer, cancer of the thymus, colon cancer, thyroid cancer, liver cancer, hepatocellular carcinoma (HCC) or sporadic or hereditary papillary renal cell carcinoma (PRCC), or any combination thereof.

In one embodiment, lung cancer is non-small cell lung cancer (NSCLC), small cell lung cancer (SCLC) or lung adenocarcinoma, pulmonary sarcomatoid carcinoma or any combination thereof.

In one embodiment, the method comprises further administration of one or more anti-cancer therapies to the subject.

In one embodiment, the one or more anti-cancer therapies comprises chemotherapy, radiation therapy, surgery, a targeted anti-cancer therapy, a kinase inhibitor, or any combination thereof.

In one embodiment, the kinase inhibitor is an inhibitor of EGFR, an inhibitor of c-Met, an inhibitor of HER2, an inhibitor of HERS, an inhibitor of HER4, an inhibitor of VEGFR or an inhibitor of AXL.

In one embodiment, the kinase inhibitor is erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib.

In one embodiment, the bispecific anti-EGFR/c-Met antibody is administered at a dose of between about 140 mg to about 2240 mg.

In one embodiment, the bispecific anti-EGFR/c-Met antibody is administered at a dose of about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1575 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200, or about 2240 mg.

In one embodiment, the bispecific anti-EGFR/c-Met antibody is administered at a dose of 1050 mg.

In one embodiment, the bispecific anti-EGFR/c-Met antibody is administered at a dose of 1400 mg.

In one embodiment, the bispecific anti-EGFR/c-Met antibody is administered twice a week, once a week, once in two weeks, once in three weeks or once in four weeks.

In one embodiment, the bispecific anti-EGFR/c-Met antibody is administered at a dose of 1575 mg.

In one embodiment, the bispecific anti-EGFR/c-Met antibody is administered at a dose of 1600 mg.

In one embodiment, the bispecific anti-EGFR/c-Met antibody is administered at a dose of 2100 mg.

In one embodiment, the bispecific anti-EGFR/c-Met antibody is administered at a dose of 2240 mg.

In one embodiment, the bispecific anti-EGFR/c-Met antibody is administered twice a week, once a week, once in two weeks, once in three weeks or once in four weeks.

In one embodiment, the bispecific anti-EGFR/c-Met antibody is administered intravenously.

In one embodiment, the bispecific anti-EGFR/c-Met antibody is administered subcutaneously.

### BRIEF DESCRIPTION OF THE DRAWINGS

FIGS. 1A-1B show receptor expression, as IHC score, plotted versus signaling, as PLA score for EGFR (FIG. 1A) and MET (FIG. 1B).

FIG. 2A-2C show representative in vivo efficacy plots of amivantamab, Fc-silent EGFR/MET, or isotype control in mouse xenograft tumors for LXFA677 (FIG. 2A), LXFA1584 (FIG. 2B), and LXFA2158 (FIG. 2C).

FIG. 3A-3D show amivantamab efficacy as % tumor growth inhibition (% TGI)) in selected PDX models having EGFR lacking activating mutations, plotted in relation to EGFR IHC H-scores (FIG. 3A) and PLA scores (FIG. 3B), and MET IHC H-scores (FIG. 3C) and PLA scores (FIG. 3D).

FIG. 4A-4C show expression (FIG. 4A) and mutational status of common oncogenes (FIG. 4B) in PDX models having EGFR lacking activating mutations, in which efficacy was tested, shown as % tumor growth inhibition (% TGI)) (FIG. 4C). The arrows indicate models with mutations in the KRAS or PI3K pathways.

FIG. 5A-5B show correlation plots of amivantamab efficacy, shown as % tumor growth inhibition (% TGI) versus combined IHC H-score and PLA score (IHC+PLA) for EGFR (FIG. 5A) and MET (FIG. 5B) in select models.

FIG. 6 shows correlation plots of amivantamab efficacy, shown as % tumor growth inhibition (% TGI) versus the expression of the EGFR ligand amphiregulin (AREG), as measured by RNA-Seq using Transcripts Per Kilobase Million (TPM).

### DETAILED DESCRIPTION

### Definitions

All publications, including but not limited to patents and patent applications, cited in this specification are herein incorporated by reference as though fully set forth.

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

“Isolated” refers to a homogenous population of molecules (such as synthetic polynucleotides, polypeptides vectors or viruses) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Newly diagnosed” refers to a subject who has been diagnosed with EGFR or c-Met expressing cancer but has not yet received treatment for multiple myeloma.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Subject” includes any human or nonhuman animal “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Cancer” refers to an abnormal growth of cells which tend to proliferate in an uncontrolled way and, in some cases, to metastasize (spread) to other areas of a patient's body.

“EGFR or c-Met expressing cancer” refers to cancer that has detectable expression of EGFR or c-Met or has EGFR or c-Met mutation or amplification. EGFR or c-Met expression, amplification and mutation status can be detected using know methods, such as sequencing, fluorescent in situ hybridization, immunohistochemistry, flow cytometry or western blotting.

“Epidermal growth factor receptor” or “EGFR” refers to the human EGFR (also known as HER1 or ErbB1 (Ullrich et al., Nature 309:418-425, 1984) having the amino acid sequence shown in UniProt identifier: P00533-1 (SEQ ID NO: 21), as well as naturally-occurring variants or mutants thereof.

SEQ ID NO: 21:
MRPSGTAGAALLALLAALCPASRALEEKKVCQGTSNKLTQLGTFEDHFL
SLQRMFNNCEVVLGNLEITYVQRNYDLSFLKTIQEVAGYVLIALNTVER
IPLENLQIIRGNMYYENSYALAVLSNYDANKTGLKELPMRNLQEILHGA
VRFSNNPALCNVESIQWRDIVSSDFLSNMSMDFQNHLGSCQKCDPSCPN
GSCWGAGEENCQKLTKIICAQQCSGRCRGKSPSDCCHNQCAAGCTGPRE
SDCLVCRKFRDEATCKDTCPPLMLYNPTTYQMDVNPEGKYSFGATCVKK
CPRNYVVTDHGSCVRACGADSYEMEEDGVRKCKKCEGPCRKVCNGIGIG
EFKDSLSINATNIKHFKNCTSISGDLHILPVAFRGDSFTHTPPLDPQEL
DILKTVKEITGFLLIQAWPENRTDLHAFENLEIIRGRTKQHGQFSLAVV
SLNITSLGLRSLKEISDGDVIISGNKNLCYANTINWKKLFGTSGQKTKI
ISNRGENSCKATGQVCHALCSPEGCWGPEPRDCVSCRNVSRGRECVDKC
NLLEGEPREFVENSECIQCHPECLPQAMNITCTGRGPDNCIQCAHYIDG
PHCVKTCPAGVMGENNTLVWKYADAGHVCHLCHPNCTYGCTGPGLEGCP
TNGPKIPSIATGMVGALLLLLVVALGIGLFMRRRHIVRKRTLRRLLQER
ELVEPLTPSGEAPNQALLRILKETEFKKIKVLGSGAFGTVYKGLWIPEG
EKVKIPVAIKELREATSPKANKEILDEAYVMASVDNPHVCRLLGICITS
TVQLITQLMPFGCLLDYVREHKDNIGSQYLLNWCVQIAKGMNYLEDRRL
VHRDLAARNVLVKTPQHVKITDFGLAKLLGAEEKEYHAEGGKVPIKWMA
LESILHRIYTHQSDVWSYGVTVWELMTFGSKPYDGIPASEISSILEKGE
RLPQPPICTIDVYMIMVKCWMIDADSRPKFRELIIEFSKMARDPQRYLV
IQGDERMHLPSPTDSNFYRALMDEEDMDDVVDADEYLIPQQGFFSSPST
SRTPLLSSLSATSNNSTVACIDRNGLQSCPIKEDSFLQRYSSDPTGALT
EDSIDDTFLPVPEYINQSVPKRPAGSVQNPVYHNQPLNPAPSRDPHYQD
PHSTAVGNPEYLNTVQPTCVNSTFDSPAHWAQKGSHQISLDNPDYQQDF
FPKEAKPNGIFKGSTAENAEYLRVAPQSSEFIGA

“Hepatocyte growth factor receptor” or “c-Met” as used herein refers to the human c-Met having the amino acid sequence shown in GenBank Accession No: NP_001120972 and natural variants thereof.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Complementarity determining regions” (CDR) are antibody regions that bind an antigen. CDRs may be defined using various delineations such as Kabat (Wu et al. (1970) J Exp Med 132: 211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton (1996) J Bmol Biol 263: 800-15). The correspondence between the various delineations and variable region numbering are described (see e.g. Lefranc et al. (2003) Dev Comp Immunol 27: 55-77; Honegger and Pluckthun, (2001) J Mol Biol 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Recombinant” refers to DNA, antibodies and other proteins that are prepared, expressed, created or isolated by recombinant means when segments from different sources are joined to produce recombinant DNA, antibodies or proteins.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“PD-(L)1 axis inhibitor” refers to a molecule that inhibits PD-1 downstream signaling. PD-(L)1 axis inhibitor may be a molecule that binds PD-1, PD-L1 or PD-L2.

“Biological sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, tumor tissue biopsies, tumor tissue samples, fine needle aspirations, surgically resected tissue, organ cultures or cell cultures.

“Low fucose” or “low fucose content” as used in the application refers to antibodies with fucose content of about between 1%-15%.

“Normal fucose” or ‘normal fucose content” as used herein refers to antibodies with fucose content of about over 50%, typically about over 80% or over 85%.

“Silent Fe” as used herein refers an Fc domain, that has been modified to have a decreased binding to an Fcγ receptor (FcγR) or decreased effector function, such as ADCC, ADCP and/or CDC, as compared to the non-modified Fc. The modifications in the Fc may be mutations in positions 214, 233, 234, 235, 236, 237, 238, 265, 267, 268, 270, 295, 297, 309, 327, 328, 329, 330, 331 or 365. Exemplary mutations that may be made singularly or in combination are mutations K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination mutations that result in antibodies with reduced ADCC are mutations L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P3315 on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Exemplary mutation that result in antibodies with reduced CDC is a K322A mutation. Residue numbering is according to the EU numbering (see e.g. IMGT® Web resources; IMGT® Repertoire (IG and TR); Proteins and alleles; allotypes).

### Methods of the Disclosure

Amivantamab or JNJ-61186372 (JNJ-372) is an IgG1 anti-EGFR/c-Met bispecific antibody described in U.S. Pat. No. 9,593,164.

The disclosure is based, at least in part, on the finding that amivantamab is effective in treating tumors having EGFR lacking activating mutations.

EGFR activating mutations that may be associated with cancer include point mutations, deletion mutations, insertion mutations, inversions or gene amplifications that lead to an increase in at least one biological activity of EGFR, such as elevated tyrosine kinase activity, enhanced ligand binding, ligand-independent signaling, increased cell proliferation, signaling to MAPK/ERK pathways, gene transcription, formation of receptor homodimers and heterodimers, dimerization (EGFR:EGFR), heterodimerization (EGFR:HER2 or EGFR:HER3). Mutations can be located in any portion of an EGFR gene or regulatory region associated with an EGFR gene and include mutations in exon 18, 19, 20 or 21 or mutations in the kinase domain. Other examples of EGFR activating mutations are known in the art (see e.g., U.S. Pat. Publ. No. US2005/0272083). Information about EGFR and other ErbB receptors including receptor homo- and hetero-dimers, receptor ligands, autophosphorylation sites, and signaling molecules involved in ErbB mediated signaling is known in the art (see e.g., Hynes and Lane, Nature Reviews Cancer 5: 341-354, 2005).

In some embodiments, the EGFR activating mutation comprises L718Q, G719A, G719X (X being any amino acid), L861X (X being any amino acid), L858R, E746K, L747S, E749Q, A750P, A755V, V765M, C797S, L858P or T790M substitution, deletion of E746-A750, deletion of R748-P753, insertion of Ala (A) between M766 and A767, insertion of Ser, Val and Ala (SVA) between S768 and V769, insertion of Asn and Ser (NS) between P772 and H773, insertion of one or more amino acids between D761 and E762, A763 and Y764, Y764 and Y765, M766 and A767, A767 and V768, S768 and V769, V769 and D770, D770 and N771, N771 and P772, P772 and H773, H773 and V774, V774 and C775, one or more deletions in EGFR exon 20, or one or more insertions in EGFR exon 20, or any combination thereof. Subjects with EGFR exon 20 mutations (insertion of one or more amino acids) are generally resistant to EGFR tyrosine kinase inhibitors (TKI) (see. e.g. Int. Pat. Publ. No. WO2018/094225).

In some embodiments, the EGFR activating mutation comprises one or more uncommon EGFR activating mutations such as S768I, L861Q and G719X.

EGFR mutation status can be detected using methods known in the art, such as for example Sanger sequencing, next-generation sequencing (NGS), whole exome sequencing (WES), RNA-Seq, fluorescent in situ hybridization, or immunohistochemistry.

The disclosure provides a method of treating a subject having cancer that lacks EGFR-activating mutations, comprising administering a therapeutically effective amount of an isolated bispecific anti-epidermal growth factor receptor (EGFR)/hepatocyte growth factor receptor (c-Met) antibody to the subject having cancer that is positive for EGFR lacking activating mutations.

The disclosure also provides a method of treating a subject having lung cancer that lacks EGFR-activating mutations, comprising administering a therapeutically effective amount of an isolated bispecific anti-EGFR/c-Met antibody to the subject having lung cancer that lacks EGFR-activating mutations.

The disclosure also provides a method of treating a subject having non-small cell lung cancer (NSCLC) that lacks EGFR-activating mutations, comprising administering a therapeutically effective amount of an isolated bispecific anti-EGFR/c-Met antibody to the subject having NSCLC that lacks EGFR-activating mutations.

The disclosure also provides a method of treating a subject having small cell lung cancer (SCLC) that lacks EGFR-activating mutations, comprising administering a therapeutically effective amount of an isolated bispecific anti-EGFR/c-Met antibody to the subject having SCLC that lacks EGFR-activating mutations.

The disclosure also provides a method of treating a subject having lung adenocarcinoma that is positive for EGFR lacking activating mutations, comprising administering a therapeutically effective amount of an isolated bispecific anti-EGFR/c-Met antibody to the subject having lung adenocarcinoma that is positive for EGFR lacking activating mutations.

The disclosure also provides a method of treating a subject having cancer with a bispecific anti-EGFR/c-Met antibody, comprising:
providing a biological sample from the subject;
determining presence or absence of a EGFR lacking activating mutations in the sample;
administering or providing for administration the bispecific anti-EGFR/c-Met antibody to the subject determined to have EGFR lacking activating mutations.

In some embodiments, the biological sample is a blood sample.

In some embodiments, the biological sample is a tumor tissue biopsy.

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the first domain that specifically binds EGFR comprises a heavy chain variable region (VH) of SEQ ID NO: 13 and a light chain variable region (VL) of SEQ ID NO: 14; and the second domain that specifically binds c-Met comprises the VH of SEQ ID NO: 15 and the VL of SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments, the bispecific anti-EGFR/c-Met antibody comprises a biantennary glycan structure with a fucose content of about between 1% to about 15%.

Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64(:249-65, 2012), application of a variant CHO line Lec13 as the host cell line (Shields et al., J Biol Chem 277:26733-26740, 2002), application of a variant CHO line EB66 as the host cell line (Olivier et al., MAbs ;2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α1,6-fucosyltrasferase (FUT8) gene (Mori et al., Biotechnol Bioeng88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem281:5032-5036, 2006, Ferrara et al., Biotechnol Bioeng 93:851-861, 2006; Xhou et al., Biotechnol Bioeng 99:652-65, 2008). In general, lowering fucose content in the glycan of the antibodies potentiates antibody-mediated cellular cytotoxicity (ADCC).

The disclosure also provides a method of treating a subject having cancer that lacks EGFR-activating mutations, comprising administering a therapeutically effective amount of an isolated bispecific anti-EGFR/c-Met antibody to the subject having cancer that is positive for EGFR lacking activating mutations, wherein the bispecific anti-EGFR/c-Met antibody comprises a first domain that specifically binds EGFR and a second domain that specifically binds c-Met, wherein the first domain comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

The disclosure also provides a method of treating a subject having lung cancer that lacks EGFR-activating mutations, comprising administering a therapeutically effective amount of an isolated bispecific anti-EGFR/c-Met antibody to the subject having lung cancer that that lacks EGFR-activating mutations, wherein the bispecific anti-EGFR/c-Met antibody comprises a first domain that specifically binds EGFR and a second domain that specifically binds c-Met, wherein the first domain comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

The disclosure also provides a method of treating a subject having NSCLC that that lacks EGFR-activating mutations, comprising administering a therapeutically effective amount of an isolated bispecific anti-EGFR/c-Met antibody to the subject having NSCLC that that lacks EGFR-activating mutations, wherein the bispecific anti-EGFR/c-Met antibody comprises a first domain that specifically binds EGFR and a second domain that specifically binds c-Met, wherein the first domain comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

The disclosure also provides a method of treating a subject having SCLC that lacks EGFR-activating mutations, comprising administering a therapeutically effective amount of an isolated bispecific anti-EGFR/c-Met antibody to the subject having SCLC that that lacks EGFR-activating mutations, wherein the bispecific anti-EGFR/c-Met antibody comprises a first domain that specifically binds EGFR and a second domain that specifically binds c-Met, wherein the first domain comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

The disclosure also provides a method of treating a subject having lung adenocarcinoma that that lacks EGFR-activating mutations, comprising administering a therapeutically effective amount of an isolated bispecific anti-EGFR/c-Met antibody to the subject having lung adenocarcinoma that that lacks EGFR-activating mutations, wherein the bispecific anti-EGFR/c-Met antibody comprises a first domain that specifically binds EGFR and a second domain that specifically binds c-Met, wherein the first domain comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

The disclosure provides a method of treating a subject having cancer that lacks EGFR-activating mutations, comprising administering a therapeutically effective amount of an isolated bispecific anti-EGFR/c-Met antibody to the subject having cancer that lacks EGFR-activating mutations, wherein the bispecific anti-EGFR/c-Met antibody comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure also provides a method of treating a subject having lung cancer that lacks EGFR-activating mutations, comprising administering a therapeutically effective amount of an isolated bispecific anti-EGFR/c-Met antibody to the subject having lung cancer that lacks EGFR-activating mutations, wherein the bispecific anti-EGFR/c-Met antibody comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure also provides a method of treating a subject having NSCLC that lacks EGFR-activating mutations, comprising administering a therapeutically effective amount of an isolated bispecific anti-EGFR/c-Met antibody to the subject having NSCLC that lacks EGFR-activating mutations, wherein the bispecific anti-EGFR/c-Met antibody comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure also provides a method of treating a subject having SCLC that lacks EGFR-activating mutations, comprising administering a therapeutically effective amount of an isolated bispecific anti-EGFR/c-Met antibody to the subject having SCLC that is positive for EGFR lacking activating mutations, wherein the bispecific anti-EGFR/c-Met antibody comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure also provides a method of treating a subject having lung adenocarcinoma that lacks EGFR-activating mutations, comprising administering a therapeutically effective amount of an isolated bispecific anti-EGFR/c-Met antibody to the subject having lung adenocarcinoma that is positive for EGFR lacking activating mutations, wherein the bispecific anti-EGFR/c-Met antibody comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure provides a method of treating a subject having cancer that lacks EGFR-activating mutations, comprising administering a therapeutically effective amount of an isolated bispecific anti-EGFR/c-Met antibody to the subject having cancer that is positive for EGFR lacking activating mutations, wherein the bispecific anti-EGFR/c-Met antibody is an IgG1 isotype and comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure also provides a method of treating a subject having lung cancer that lacks EGFR-activating mutations, comprising administering a therapeutically effective amount of an isolated bispecific anti-EGFR/c-Met antibody to the subject having lung cancer that is positive for EGFR lacking activating mutations, wherein the bispecific anti-EGFR/c-Met antibody is an IgG1 isotype and comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure also provides a method of treating a subject having NSCLC that lacks EGFR-activating mutations, comprising administering a therapeutically effective amount of an isolated bispecific anti-EGFR/c-Met antibody to the subject having NSCLC that is positive for EGFR lacking activating mutations, wherein the bispecific anti-EGFR/c-Met antibody is an IgG1 isotype and comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure also provides a method of treating a subject having SCLC that lacks EGFR-activating mutations, comprising administering a therapeutically effective amount of an isolated bispecific anti-EGFR/c-Met antibody to the subject having SCLC that is positive for EGFR lacking activating mutations, wherein the bispecific anti-EGFR/c-Met antibody is an IgG1 isotype and comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

The disclosure also provides a method of treating a subject having lung adenocarcinoma that lacks EGFR-activating mutations, comprising administering a therapeutically effective amount of an isolated bispecific anti-EGFR/c-Met antibody to the subject having lung adenocarcinoma that is positive for EGFR lacking activating mutations, wherein the bispecific anti-EGFR/c-Met antibody is an IgG1 isotype and comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

In some embodiments, the bispecific anti-EGFR/c-Met antibody is an IgG1 isotype. Some variation exists within the IgG1 constant domain (e.g. well-known allotypes), with variation at positions 214, 356, 358, 422, 431, 435 o 436 (residue numbering according to the EU numbering) (see e.g. IMGT Web resources; IMGT Repertoire (IG and TR); Proteins and alleles; allotypes). The bispecific anti-EGFR/c-Met antibody may be of any IgG1 allotype, such as G1m17, G1m3, G1m1,G1m2, G1m27 or G1m28.

The disclosure also provides a method of treating a subject having cancer that lacks EGFR-activating mutations, comprising administering a therapeutically effective amount of an isolated bispecific anti-EGFR/c-Met antibody to the subject having cancer that is positive for EGFR lacking activating mutations, wherein the bispecific anti-EGFR/c-Met antibody comprises a HC1 of SEQ ID NO: 17, a LC1 of SEQ ID NO: 18, a HC2 of SEQ ID NO: 19 and a LC2 of SEQ ID NO: 20.

The disclosure also provides a method of treating a subject having lung cancer that lacks EGFR-activating mutations, comprising administering a therapeutically effective amount of an isolated bispecific anti-EGFR/c-Met antibody to the subject having lung cancer that is positive for EGFR lacking activating mutations, wherein the bispecific anti-EGFR/c-Met antibody comprises a HC1 of SEQ ID NO: 17, a LC1 of SEQ ID NO: 18, a HC2 of SEQ ID NO: 19 and a LC2 of SEQ ID NO: 20.

The disclosure also provides a method of treating a subject having NSCLC that lacks EGFR-activating mutations, comprising administering a therapeutically effective amount of an isolated bispecific anti-EGFR/c-Met antibody to the subject having NSCLC that is positive for EGFR lacking activating mutations, wherein the bispecific anti-EGFR/c-Met antibody comprises a HC1 of SEQ ID NO: 17, a LC1 of SEQ ID NO: 18, a HC2 of SEQ ID NO: 19 and a LC2 of SEQ ID NO: 20.

The disclosure also provides a method of treating a subject having SCLC that lacks EGFR-activating mutations, comprising administering a therapeutically effective amount of an isolated bispecific anti-EGFR/c-Met antibody to the subject having SCLC that is positive for EGFR lacking activating mutations, wherein the bispecific anti-EGFR/c-Met antibody comprises a HC1 of SEQ ID NO: 17, a LC1 of SEQ ID NO: 18, a HC2 of SEQ ID NO: 19 and a LC2 of SEQ ID NO: 20.

The disclosure also provides a method of treating a subject having lung adenocarcinoma that lacks EGFR-activating mutations, comprising administering a therapeutically effective amount of an isolated bispecific anti-EGFR/c-Met antibody to the subject having lung adenocarcinoma that is positive for EGFR lacking activating mutations, wherein the bispecific anti-EGFR/c-Met antibody comprises a HC1 of SEQ ID NO: 17, a LC1 of SEQ ID NO: 18, a HC2 of SEQ ID NO: 19 and a LC2 of SEQ ID NO: 20.

In some embodiments, the subject is relapsed or resistant to treatment with one or more prior anti-cancer therapies.

In some embodiments, the one or more prior anti-cancer therapies comprises one or more chemotherapeutic agents, checkpoint inhibitors, targeted anti-cancer therapies or kinase inhibitors, or any combination thereof.

In some embodiments, the kinase inhibitor is an inhibitor of EGFR, an inhibitor of c-Met, an inhibitor of HER2, an inhibitor of HERS, an inhibitor of HER4, an inhibitor of VEGFR or an inhibitor of AXL.

In some embodiments, the kinase inhibitor is erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib.

In some embodiments, the one or more prior anti-cancer therapies comprises carboplatin, paclitaxel, gemcitabine, cisplatin, vinorelbine, docetaxel, palbociclib, crizotinib, PD-(L)1 axis inhibitor, an inhibitor of EGFR, an inhibitor of c-Met, an inhibitor of HER2, an inhibitor of HER3, an inhibitor of HER4, an inhibitor of VEGFR, an inhibitor of AXL, erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib, or any combination thereof.

In some embodiments, the subject is resistant or has acquired resistance to an EGFR inhibitor. Exemplary EGFR inhibitors for which cancer may acquire resistance are anti-EGFR antibodies cetuximab (ERBITUX®), pantinumumab (VECTIBIX®), matuzumab, nimotuzumab, small molecule EGFR inhibitors erlotinib (TARCEVA®), gefitinib (IRESSA®), EKB-569 (pelitinib, irreversible EGFR TKI), pan-ErbB and other receptor tyrosine kinase inhibitors, lapatinib (EGFR and HER2 inhibitor), pelitinib (EGFR and HER2 inhibitor),vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BIBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR TKI),and HKI-272 (neratinib, irreversible EGFR/ErbB2 inhibitor).

Various qualitative and/or quantitative methods may be used to determine if a subject is resistant, has developed or is susceptible to developing a resistance to treatment with an anti-cancer therapy. Symptoms that may be associated with resistance to an anti-cancer therapy include a decline or plateau of the well-being of the patient, an increase in the size of a tumor, arrested or slowed decline in growth of a tumor, and/or the spread of cancerous cells in the body from one location to other organs, tissues or cells. Re-establishment or worsening of various symptoms associated with cancer may also be an indication that a subject has developed or is susceptible to developing resistance to an anti-cancer therapy, such as anorexia, cognitive dysfunction, depression, dyspnea, fatigue, hormonal disturbances, neutropenia, pain, peripheral neuropathy, and sexual dysfunction. The symptoms associated with cancer may vary according to the type of cancer. For example, symptoms associated with cervical cancer may include abnormal bleeding, unusual heavy vaginal discharge, pelvic pain that is not related to the normal menstrual cycle, bladder pain or pain during urination, and bleeding between regular menstrual periods, after sexual intercourse, douching, or pelvic exam Symptoms associated with lung cancer may include persistent cough, coughing up blood, shortness of breath, wheezing chest pain, loss of appetite, losing weight without trying and fatigue. Symptoms for liver cancer may include loss of appetite and weight, abdominal pain, especially in the upper right part of abdomen that may extend into the back and shoulder, nausea and vomiting, general weakness and fatigue, an enlarged liver, abdominal swelling (ascites), and a yellow discoloration of the skin and the whites of eyes (jaundice). One skilled in oncology may readily identify symptoms associated with a particular cancer type.

Exemplary PD-(L)1 axis inhibitors are antibodies that bind PD-1 such as nivolumab (OPDIVO®), pembrolimumab (KEYTRUDA®), sintilimab, cemiplimab (LIBTAYO®), tripolibamab, tislelizumab, spartalizumab, camrelizumab, dostralimab, genolimzumab or cetrelimab, or antibodies that bind PD-L1, such as PD-L1 antibodies are envafolimab, atezolizumab (TECENTRIQ®), durvalumab (IMFINZI®) and avelumab (BAVENCIO®).

Marketed antibodies may be purchased via authorized distributor or pharmacy. The amino acid sequences structures of the small molecules can be found from USAN and/or INN submissions by the companies of from CAS registry.

In some embodiments, the subject is treatment naïve.

In some embodiments, cancer that is positive for EGFR lacking activating mutations is positive for CDK4 amplification, EGFR amplification, KRAS amplification, MDM2 amplification, TERT amplification, NF1 R2450*; RAD50 L597Vfs*5, MET c.3082+3A>G, increased levels of circulating HGF, c-MET amplification, or any combination thereof.

In some embodiments, cancer that is positive for the EGFR lacking activating mutations is positive for at least one mutation in a gene selected from the group consisting of ALK, APC, BRAF, BRCA1, BRCA2, CDKN2A, CDKN2B, CTNNB1, ERBB2, ERBB3, FGFR3, KIT, LRP1B, MET, MLH1, MSH3, NOTCH1, NTRK1, RET, ROS1, STK11, TP53, and VEGFA. In some embodiments, the at least one mutation is a mutation selected from the group consisting of a point mutation, a deletion mutation, an insertion mutation, an inversion, gene amplification, and gene fusion. Mutations can be located in any portion of a gene or regulatory regions associated with the gene. A mutation can be detected using methods known in the art, such as for example Sanger sequencing, next-generation sequencing (NGS), whole exome sequencing (WES), RNA-Seq, fluorescent in situ hybridization, or immunohistochemistry.

In some embodiments, the at least one mutation in APC is S2621C, N813S, E1317Q, or R549G.

In some embodiments, the at least one mutation in BRCA1 is M128V, G275S, Y179C, F486L, or N550H.

In some embodiments, the at least one mutation in BRCA2 is S326R, R2973H, R2034C, I283V, R672X, G25X, R468X, or I1929M (where X is any amino acid).

In some embodiments, the at least one mutation in CDKN2A is G23X, A100X, D84H, C72X, H83N, or G111X (where X is any amino acid).

In some embodiments, the at least one mutation in CTNNB1 is T41A.

In some embodiments, the at least one mutation in ERBB2 is R1146W, V1180X (where X is any amino acid), or A386D.

In some embodiments, the at least one mutation in ERBB3 is K998R, L1177I, or G513D.

In some embodiments, the at least one mutation in FGFR3 is G639R or E85K.

In some embodiments, the at least one mutation in LRP1B is P4512A, A3816V, T3393K, Q3636H, M1V, C1554S, S1083N, T2482S, C3522Y, G1965C, P2882T, P3372A, I1266L, L4268X (where X is any amino acid), S449T, E4352G, C864R, F1435I, D3697Y, V2033F, A3308S, S1281N, D1807E.

In some embodiments, the at least one mutation in MET is E168D.

In some embodiments, the at least one mutation in MSH3 is E1036Q,

In some embodiments, the at least one mutation in NOTCH1 is A1696V, R1279C, E1450K, Q2184R, Q2184K, T701P, or C612Y.

In some embodiments, the at least one mutation in TP53 is R280G, P278S, E198X, H193L, R379S, V172X, G245D, L194R, H179Y, L265P, R110L, R158L, R248W, I332M, G244C, R273H, Y163C, H193R, R158L, Y103X, M237I, R273L, R273H, E171X, or R249M (where X is any amino acid).

In some embodiments, the at least one mutation in VEGFA is R114W, R87W or R335C.

Exemplary c-Met activating mutations include point mutations, deletion mutations, insertion mutations, inversions or gene amplifications that lead to an increase in at least one biological activity of a c-Met protein, such as elevated tyrosine kinase activity, formation of receptor homodimers and heterodimers, enhanced ligand binding etc. Mutations can be located in any portion of the c-Met gene or regulatory regions associated with the gene, such as mutations in the kinase domain of c-Met. Exemplary c-Met activating mutations are mutations at residue positions N375, V13, V923, R175, V136, L229, 5323, R988, S1058/T1010 and E168. Methods for detecting EGFR and c-Met mutations or gene amplifications are well known.

In some embodiments, the mutant KRAS comprises a G12V, G12C, G12A, or G12D substitution, or any combination thereof.

In some embodiments, cancer that is positive for the EGFR lacking activating mutations is positive for the expression of at least one EGFR ligand. The examples of EGFR ligands include but are not limited to Epidermal growth factor (EGF), amphiregulin (AREG), transforming growth factor α (TGFα), heparin-binding EGF-like growth factor (HBEGF), betacellulin (BTC), epiregulin (EREG), and epigen (EPGN).

In some embodiments, the method of treating a cancer that is positive for the EGFR lacking activating mutations further comprises determining levels of at least one EGFR ligand, and administering or providing for administration the bispecific anti-EGFR/c-Met antibody to the subject determined to have the EGFR lacking activating mutations and determined to be positive for gene expression levels or protein levels of at least one EGFR ligand.

In some embodiments, the method of treating a cancer that is positive for the EGFR lacking activating mutations further comprises determining levels of amphiregulin , and administering or providing for administration the bispecific anti-EGFR/c-Met antibody to the subject determined to have the EGFR lacking activating mutations and determined to be positive for amphiregulin gene expression levels or protein levels. In some embodiments, the amphiregulin gene expression levels or protein levels may be compared to a control value.

In some embodiments, cancer that lacks EGFR-activating mutations comprises lung cancer, gastric cancer, colorectal cancer, brain cancer, derived from epithelial cell cancer, breast cancer, ovarian cancer, colorectal cancer, anal cancer, prostate cancer, kidney cancer, bladder cancer, head and neck cancer, pharynx cancer, cancer of the nose, pancreatic cancer, skin cancer, oral cancer, cancer of the tongue, esophageal cancer, vaginal cancer, cervical cancer, cancer of the spleen, testicular cancer, gastric cancer, cancer of the thymus, colon cancer, thyroid cancer, liver cancer, hepatocellular carcinoma (HCC) or sporadic or hereditary papillary renal cell carcinoma (PRCC), or any combination thereof. In some embodiments, cancer that lacks EGFR-activating mutations comprises lung cancer. In some embodiments, cancer that lacks EGFR-activating mutations comprises gastric cancer. In some embodiments, cancer that lacks EGFR-activating mutations comprises colorectal cancer. In some embodiments, cancer that lacks EGFR-activating mutations comprises brain cancer. In some embodiments, cancer that lacks EGFR-activating mutations comprises epithelial cell cancer. In some embodiments, cancer that lacks EGFR-activating mutations comprises breast cancer. In some embodiments, cancer that lacks EGFR-activating mutations comprises ovarian cancer. In some embodiments, cancer that lacks EGFR-activating mutations comprises colorectal cancer. In some embodiments, cancer that lacks EGFR-activating mutations comprises anal cancer. In some embodiments, cancer that lacks EGFR-activating mutations comprises prostate cancer. In some embodiments, cancer that lacks EGFR-activating mutations comprises kidney cancer. In some embodiments, cancer that lacks EGFR-activating mutations comprises bladder cancer. In some embodiments, cancer that lacks EGFR-activating mutations comprises head and neck cancer. In some embodiments, cancer that lacks EGFR-activating mutations comprises pharynx cancer. In some embodiments, cancer that lacks EGFR-activating mutations comprises cancer of the nose. In some embodiments, cancer that lacks EGFR-activating mutations comprises pancreatic cancer. In some embodiments, cancer that lacks EGFR-activating mutations comprises skin cancer. In some embodiments, cancer that lacks EGFR-activating mutations comprises oral cancer. In some embodiments, cancer that lacks EGFR-activating mutations comprises cancer of the tongue. In some embodiments, cancer that lacks EGFR-activating mutations comprises esophageal cancer. In some embodiments, cancer that lacks EGFR-activating mutations comprises vaginal cancer. In some embodiments, cancer that lacks EGFR-activating mutations comprises cervical cancer. In some embodiments, cancer that lacks EGFR-activating mutations comprises cancer of the spleen. In some embodiments, cancer that lacks EGFR-activating mutations comprises testicular cancer. In some embodiments, cancer that lacks EGFR-activating mutations comprises gastric cancer. In some embodiments, cancer that lacks EGFR-activating mutations comprises cancer of the thymus. In some embodiments, cancer that lacks EGFR-activating mutations comprises colon cancer. In some embodiments, cancer that lacks EGFR-activating mutations comprises thyroid cancer. In some embodiments, cancer that lacks EGFR-activating mutations comprises liver cancer. In some embodiments, cancer that lacks EGFR-activating mutations comprises hepatocellular carcinoma (HCC). In some embodiments, cancer that lacks EGFR-activating mutations comprises sporadic or hereditary papillary renal cell carcinoma (PRCC).

In some embodiments, NSCLC includes squamous cell carcinoma, adenocarcinoma, and large cell carcinoma. In some embodiments, cells of the NSCLC have an epithelial phenotype. In some embodiments, the NSCLC has acquired resistance to treatment with one or more EGFR inhibitors.

In some embodiments, the subject is further administered one or more anti-cancer therapies.

In some embodiments, the one or more anti-cancer therapies comprises chemotherapy, radiation therapy, surgery, a targeted anti-cancer therapy or a kinase inhibitor, or any combination thereof.

In some embodiments, the kinase inhibitor is an inhibitor of EGFR, an inhibitor of c-Met, an inhibitor of HER2, an inhibitor of HERS, an inhibitor of HER4, an inhibitor of VEGFR or an inhibitor of AXL. In some embodiments, the kinase inhibitor is an inhibitor of EGFR. In some embodiments, the kinase inhibitor is an inhibitor of c-Met. In some embodiments, the kinase inhibitor is an inhibitor of HER2. In some embodiments, the kinase inhibitor is an inhibitor of HER3. In some embodiments, the kinase inhibitor is an inhibitor of HER4. In some embodiments, the kinase inhibitor is an inhibitor of VEGFR. In some embodiments, the kinase inhibitor is an inhibitor of or AXL.

In some embodiments, the kinase inhibitor is erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib.

In some embodiments, the kinase inhibitor is erlotinib. In some embodiments, the kinase inhibitor is gefitinib. In some embodiments, the kinase inhibitor is lapatinib. In some embodiments, the kinase inhibitor is vandetanib. In some embodiments, the kinase inhibitor is afatinib. In some embodiments, the kinase inhibitor is osimertinib. In some embodiments, the kinase inhibitor is lazertinib. In some embodiments, the kinase inhibitor is poziotinib. In some embodiments, the kinase inhibitor is criotinib. In some embodiments, the kinase inhibitor is cabozantinib. In some embodiments, the kinase inhibitor is capmatinib. In some embodiments, the kinase inhibitor is axitinib. In some embodiments, the kinase inhibitor is lenvatinib. In some embodiments, the kinase inhibitor is nintedanib. In some embodiments, the kinase inhibitor is regorafenib. In some embodiments, the kinase inhibitor is pazopanib. In some embodiments, the kinase inhibitor is sorafenib. In some embodiments, the kinase inhibitor is sunitinib.

Anti-cancer therapies that may be administered in combination with the bispecific anti-EGFR/c-Met antibody in the methods of the disclosure include any one or more of the chemotherapeutic drugs or other anti-cancer therapeutics known to those of skill in the art. Chemotherapeutic agents are chemical compounds useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL® docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA®), afatinib (BIBW 2992), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et aL, eds.), Chapter 10, McMillan Publishing, are also applicable to the methods of the present invention.

### Administration

The bispecific anti-EGFR/c-Met antibody may be administered in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, See especially pp. 958-989.

The mode of administration may be any suitable route that delivers the bispecific anti-EGFR-c-Met antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intratumoral, intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery. In some embodiments, the bispecific anti-EGFR/c-Met antibody is administered intravenously (IV). In some embodiments, the bispecific anti-EGFR/c-Met antibody is administered subcutaneously (SC). In some embodiments, the bispecific anti-EGFR/c-Met antibody is administered using the on-body delivery device.

In some embodiments, the bispecific anti-EGFR/c-Met antibody is administered at a dose of between about 140 mg to about 2240 mg. In some embodiments, the bispecific anti-EGFR/c-Met antibody is administered at a dose of between about 140 mg to about 2240 mg.

In some embodiments, the bispecific anti-EGFR/c-Met antibody is administered at a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1575 mg, about 1580 mg, about 1590 mg, about 1600 mg, about 1610 mg, 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2130 mg, about 2140 mg, about 2150 mg, about 2160 mg, about 2170 mg, about 2180 mg, about 2190 mg, about 2200 mg, about 2210 mg, about 2220 mg, about 2230 mg, about 2240 mg, about 2250 mg, about 2260 mg, about 2270 mg, about 2280 mg, about 2290 mg, about 2300 mg.

In some embodiments, the bispecific anti-EGFR/c-Met antibody is administered at a dose of about 350 mg, about 700 mg, about 1050 mg, about 1400 mg, about 1575 mg, about 1600, about 2100 mg, or about 2240 mg. In some embodiments, the bispecific anti-EGFR/c-Met antibody is administered at a dose of about 350 mg. In some embodiments, the bispecific anti-EGFR/c-Met antibody is administered at a dose of about 700 mg. In some embodiments, the bispecific anti-EGFR/c-Met antibody is administered at a dose of about 750 mg. In some embodiments, the bispecific anti-EGFR/c-Met antibody is administered at a dose of about 800 mg. In some embodiments, the bispecific anti-EGFR/c-Met antibody is administered at a dose of about 850 mg. In some embodiments, the bispecific anti-EGFR/c-Met antibody is administered at a dose of about 900 mg. In some embodiments, the bispecific anti-EGFR/c-Met antibody is administered at a dose of about 950 mg. In some embodiments, the bispecific anti-EGFR/c-Met antibody is administered at a dose of about 1000 mg. In some embodiments, the bispecific anti-EGFR/c-Met antibody is administered at a dose of about 1050 mg. In some embodiments, the bispecific anti-EGFR/c-Met antibody is administered at a dose of about 1100 mg. In some embodiments, the bispecific anti-EGFR/c-Met antibody is administered at a dose of about 1150 mg. In some embodiments, the bispecific anti-EGFR/c-Met antibody is administered at a dose of about 1200 mg. In some embodiments, the bispecific anti-EGFR/c-Met antibody is administered at a dose of about 1250 mg. In some embodiments, the bispecific anti-EGFR/c-Met antibody is administered at a dose of about 1300 mg. In some embodiments, the bispecific anti-EGFR/c-Met antibody is administered at a dose of about 1350 mg. In some embodiments, the bispecific anti-EGFR/c-Met antibody is administered at a dose of about 1400 mg. In some embodiments, the bispecific anti-EGFR/c-Met antibody is administered at a dose of about 1575 mg. In some embodiments, the bispecific anti-EGFR/c-Met antibody is administered at a dose of about 1600 mg. In some embodiments, the bispecific anti-EGFR/c-Met antibody is administered at a dose of about 2100 mg. In some embodiments, the bispecific anti-EGFR/c-Met antibody is administered at a dose of about 2240 mg.

In some embodiments, the bispecific anti-EGFR/c-Met antibody is administered once a week. In some embodiments, the bispecific anti-EGFR/c-Met antibody is administered about 1050 mg once a week. In some embodiments, the bispecific anti-EGFR/c-Met antibody is administered about 1400 mg once a week. In some embodiments, the bispecific anti-EGFR/c-Met antibody is administered about 1575 mg once a week. In some embodiments, the bispecific anti-EGFR/c-Met antibody is administered about 1600 mg once a week. In some embodiments, the bispecific anti-EGFR/c-Met antibody is administered about 2100 mg once a week. In some embodiments, the bispecific anti-EGFR/c-Met antibody is administered about 2240 mg once a week.

In some embodiments, the bispecific anti-EGFR/c-Met antibody is administered once in two weeks. In some embodiments, the bispecific anti-EGFR/c-Met antibody is administered about 1050 mg once in two weeks. In some embodiments, the bispecific anti-EGFR/c-Met antibody is administered about 1400 mg once in two weeks. In some embodiments, the bispecific anti-EGFR/c-Met antibody is administered about 1575 mg once in two weeks. In some embodiments, the bispecific anti-EGFR/c-Met antibody is administered about 1600 mg once in two weeks. In some embodiments, the bispecific anti-EGFR/c-Met antibody is administered about 2100 mg once in two weeks. In some embodiments, the bispecific anti-EGFR/c-Met antibody is administered about 2240 mg once in two weeks.

In some embodiments, the bispecific anti-EGFR/c-Met antibody is administered twice a week. In some embodiments, the bispecific anti-EGFR/c-Met antibody is administered once a week. In some embodiments, the bispecific anti-EGFR/c-Met antibody is administered once in two weeks. In some embodiments, the bispecific anti-EGFR/c-Met antibody is administered once in three weeks. In some embodiments, the bispecific anti-EGFR/c-Met antibody is administered once in four weeks.

For combination therapies, the one or more anti-cancer agents may be administered using recommended doses and dosages of the anti-cancer agent.

### Generation of Bispecific Anti-EGFR/c-Met Antibodies Used in the Methods of the Disclosure

An exemplary bispecific anti-EGFR/c-Met antibody that can be used in the methods of the disclosures is amivantamab. Amivantamab is characterized by following amino acid sequences:

EGFR binding arm

>SEQ ID NO: 1 (HCDR1, EGFR binding arm)
TYGMH
>SEQ ID NO: 2 (HCDR2, EGFR binding arm)
VIWDDGSYKYYGDSVKG
>SEQ ID NO: 3 (HCDR3, EGFR binding arm)
DGITMVRGVMKDYFDY
>SEQ ID NO: 4 (LCDR1, EGFR binding arm)
RASQDISSALV
>SEQ ID NO: 5 (LCDR2, EGFR binding arm)
DASSLES
>SEQ ID NO: 6 (LCDR3, EGFR binding arm)
QQFNSYPLT
>SEQ ID NO: 7 (HCDR1, c-Met binding arm)
SYGIS
>SEQ ID NO: 8 (HCDR2, c-Met binding arm)
WISAYNGYTNYAQKLQG
>SEQ ID NO: 9 (HCDR3, c-Met binding arm)
DLRGTNYFDY
>SEQ ID NO: 10 (LCDR1, c-Met binding arm)
RASQGISNWLA
>SEQ ID NO: 11 (LCDR2, c-Met binding arm)
AASSLLS
>SEQ ID NO: 12 (LCDR3, c-Met binding arm)
QQANSFPIT
>SEQ ID NO: 13 (VH, EGFR binding arm)
QVQLVESGGGVVQPGRSLRLSCAASGFTFSTYGMHWVRQAPGKGLEWVAVIWDDGSYK
YYGDSVKGRFTISRDNSKNTLYLQMNSLRAEDTAVYYCARDGITMVRGVMKDYFDYWG
QGTLVTVSS
>SEQ ID NO: 14 (VL, EGFR binding arm)
AIQLTQSPSSLSASVGDRVTITCRASQDISSALVWYQQKPGKAPKLLIYDASSLESGVPSRFS
GSESGTDFTLTISSLQPEDFATYYCQQFNSYPLTFGGGTKVEIK
>SEQ ID NO: 15 (VH, c-Met binding arm)
QVQLVQSGAEVKKPGASVKVSCETSGYTFTSYGISWVRQAPGHGLEWMGWISAYNGYTN
YAQKLQGRVTMTTDTSTSTAYMELRSLRSDDTAVYYCARDLRGTNYFDYWGQGTLVTVS
S
>SEQ ID NO: 16 (VL, c-Met binding arm)
DIQMTQSPSSVSASVGDRVTITCRASQGISNWLAWFQHKPGKAPKLLIYAASSLLSGVPSRF
SGSGSGTDFTLTISSLQPEDFATYYCQQANSFPITFGQGTRLEIK
>SEQ ID NO: 17 HC1
QVQLVESGGGVVQPGRSLRLSCAASGFTFSTYGMHWVRQAPGKGLEWVAVIWDDGSY
KYYGDSVKGRFTISRDNSKNTLYLQMNSLRAEDTAVYYCARDGITMVRGVMKDYFDY
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSD
GSFLLYSKLTVDKSRWQQGNVFSCSVMHEALHNHYTQKSLSLSPGK
>SEQ ID NO: 18 LC1
AIQLTQSPSSLSASVGDRVTITCRASQDISSALVWYQQKPGKAPKLLIYDASSLESGVPSR
FSGSESGTDFTLTISSLQPEDFATYYCQQFNSYPLTFGGGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
>SEQ ID NO: 19 HC2
QVQLVQSGAEVKKPGASVKVSCETSGYTFTSYGISWVRQAPGHGLEWMGWISAYNGY
TNYAQKLQGRVTMTTDTSTSTAYMELRSLRSDDTAVYYCARDLRGTNYFDYWGQGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSRL
TVDKSRWQQGNVFSCSVMHEALHNHYTQKSLSLSPGK
>SEQ ID NO: 20 LC2
DIQMTQSPSSVSASVGDRVTITCRASQGISNWLAWFQHKPGKAPKLLIYAASSLLSGVPS
RFSGSGSGTDFTLTISSLQPEDFATYYCQQANSFPITFGQGTRLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC

Other bispecific anti-EGFR/c-Met antibodies publicly available may also be used in the methods of the disclosure as long as they demonstrate similar characteristics when compared to amivantamab as described in U.S. Pat. No. 9,593,164. Bispecific anti-EGFR/c-Met antibodies that may be used in the methods of the disclosure may also be generated by combining EGFR binding VH/VL domains and c-Met binding VH/VL domains that are publicly available and testing the resulting bispecific antibodies for their characteristics as described in U.S. Pat. No. 9,593,164.

Bispecific anti-EGFR/c-Met antibodies used in the methods of the disclosure may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e. an epitope on EGFR and an epitope on c-Met. For example, the bispecific antibodies of the invention may be generated using the technology described in Int. Pat. Publ. No. WO2011/131746. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, first monospecific bivalent antibody and the second monospecific bivalent antibody are engineered to have the aforementioned mutation in the Fc region, the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using designs such as the Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), and the Biclonic (Merus).

In the “knob-in-hole” strategy (see, e.g., Intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

CrossMAb technology, in addition to utilizing the “knob-in-hole” strategy to promoter Fab arm exchange utilizes CH1/CL domain swaps in one half arm to ensure correct light chain pairing of the resulting bispecific antibody (see e.g. U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by following substitutions (expressed as modified positions in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### Embodiments

1) A method of treating a subject having a cancer that is positive for an EGFR and lacks an at least one EGFR-activating mutation, comprising administering a therapeutically effective amount of an isolated bispecific anti-epidermal growth factor receptor (EGFR)/hepatocyte growth factor receptor (c-Met) antibody to the subject having cancer that is positive for the EGFR and lacks an at least one EGFR-activating mutation.
2) A method of treating a subject having a cancer with a bispecific anti-EGFR/c-Met antibody, comprising:
   a) providing a biological sample from the subject;
   b) determining presence or absence of an EGFR lacking activating mutations in the sample;
   c) administering or providing for administration the bispecific anti-EGFR/c-Met antibody to the subject determined to lack an EGFR-activating mutation.
3) The method of embodiment 1 or 2, wherein the at least one activating mutation is a mutation which increases at least one biological activity of EGFR.
4) The method of embodiment 3, wherein the at least one biological activity of EGFR is selected from the group consisting of tyrosine kinase activity, ligand-independent signaling, increased cell proliferation, signaling to MAPK/ERK pathways, gene transcription, dimerization (EGFR:EGFR), and heterodimerization (EGFR:HER2 or EGFR:HER3).
5) The method of embodiment 3, wherein the at least one activating mutation which increase at least one biological activity of EGFR comprise at least one mutation selected from the group consisting of L718Q, G719A, G719X (X being any amino acid), L861X (X being any amino acid), L858R, E746K, L747S, E749Q, A750P, A755V, V765M, C797S, L858P or T790M substitution, deletion of E746-A750, deletion of R748-P753, insertion of Ala (A) between M766 and A767, insertion of Ser, Val and Ala (SVA) between S768 and V769, insertion of Asn and Ser (NS) between P772 and H773, insertion of one or more amino acids between D761 and E762, A763 and Y764, Y764 and Y765, M766 and A767, A767 and V768, S768 and V769, V769 and D770, D770 and N771, N771 and P772, P772 and H773, H773 and V774, V774 and C775, one or more deletions in EGFR exon 20, one or more insertions in EGFR exon 20, S768I, L861Q and G719X (X being any amino acid).
6) The method of any one of embodiments 1-5, wherein the method further comprises determining presence or absence of at least one mutation in any one gene selected from the group consisting of KRAS, PIK3CA, and PTEN, and administering or providing for administration the bispecific anti-EGFR/c-Met antibody to the subject determined to have the EGFR lacking activating mutations and determined to lack at least one mutation in any one gene selected from the group consisting of KRAS, PIK3CA, and PTEN.
7) The method of embodiment 6 wherein the at least one mutation in KRAS is selected from the group consisting of G12V, G12C, G12A and G12D.
8) The method of embodiment 7 wherein the at least one mutation in KRAS is G12C.
9) The method of embodiment 8 wherein the at least one mutation in PI3K is selected from the group consisting of E545K, H1047L, and PI3K amplification.
10) The method of embodiment 6 wherein the at least one mutation in PTEN is PTEN deletion.
11) The method of embodiments 1-10, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.
12) The method of any one of embodiments 1-11, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.
13) The method of any one of embodiments 1-12, wherein the bispecific anti-EGFR/c-Met antibody is an IgG1 isotype.
14) The method of any one of embodiments 1-13, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.
15) The method of any one of embodiments 1-14, wherein the bispecific anti-EGFR/c-Met antibody comprises one or more Fc silencing mutations.
16) The method of embodiment 14 wherein the one or more Fc silencing mutations decrease affinity to Fcγ receptors.
17) The method of embodiment 15 or 16 wherein the one or more Fc silencing mutations comprise V234A/G237A/P238S/H268A/V309L/A330S/P331S.
18) The method of any one of embodiments 1-17, wherein the bispecific anti-EGFR/c-Met antibody comprises a biantennary glycan structure with a fucose content between about 1% to about 15%.
19) The method of any one of embodiments 1-18, wherein the subject is relapsed or resistant to treatment with one or more prior anti-cancer therapies.
20) The method of embodiment 19, wherein the one or more prior anti-cancer therapies comprises one or more chemotherapeutic agents, checkpoint inhibitors, targeted anti-cancer therapies or kinase inhibitors, or any combination thereof.
21) The method of embodiment 20, wherein the one or more prior anti-cancer therapies comprises carboplatin, paclitaxel, gemcitabine, cisplatin, vinorelbine, docetaxel, palbociclib, crizotinib, PD-(L)1 axis inhibitor, an inhibitor of EGFR, an inhibitor of c-Met, an inhibitor of HER2, an inhibitor of HER3, an inhibitor of HER4, an inhibitor of VEGFR, an inhibitor of AXL, erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib, or any combination thereof.
22) The method of any one of embodiments 1-18, wherein the subject is treatment naïve.
23) The method of any one of embodiments 1-22, wherein cancer that is positive for the EGFR lacking activating mutations is positive for at least one mutation in a gene selected from the group consisting of ALK, APC, BRAF, BRCA1, BRCA2, CDKN2A, CDKN2B, CTNNB1, ERBB2, ERBB3, FGFR3, KIT, LRP1B, MET, MLH1, MSH3, NOTCH1, NTRK1, RET, ROS1, STK11, TP53, and VEGFA.
24) The method of any one of embodiments 1-23, wherein the cancer is lung cancer, gastric cancer, colorectal cancer, brain cancer, cancer derived from epithelial cells, breast cancer, ovarian cancer, colorectal cancer, anal cancer, prostate cancer, kidney cancer, bladder cancer, head and neck cancer, pharynx cancer, cancer of the nose, pancreatic cancer, skin cancer, oral cancer, cancer of the tongue, esophageal cancer, vaginal cancer, cervical cancer, cancer of the spleen, testicular cancer, gastric cancer, cancer of the thymus, colon cancer, thyroid cancer, liver cancer, hepatocellular carcinoma (HCC) or sporadic or hereditary papillary renal cell carcinoma (PRCC), or any combination thereof.
25) The method of embodiment 24, wherein lung cancer is non-small cell lung cancer (NSCLC), small cell lung cancer (SCLC) or lung adenocarcinoma, pulmonary sarcomatoid carcinoma or any combination thereof.
26) The method of any one of embodiments 1-25, comprising further administering one or more anti-cancer therapies to the subject.
27) The method of embodiment 26, wherein the one or more anti-cancer therapies comprises chemotherapy, radiation therapy, surgery, a targeted anti-cancer therapy, a kinase inhibitor, or any combination thereof.
28) The method of embodiment 20, wherein the kinase inhibitor is an inhibitor of EGFR, an inhibitor of c-Met, an inhibitor of HER2, an inhibitor of HER3, an inhibitor of HER4, an inhibitor of VEGFR or an inhibitor of AXL.
29) The method of embodiment 28, wherein the kinase inhibitor is erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib.
30) The method of any one of embodiments 1-29, wherein the bispecific anti-EGFR/c-Met antibody is administered at a dose of between about 140 mg to about 2240 mg.
31) The method of any one of embodiments 1-30, wherein the bispecific anti-EGFR/c-Met antibody is administered at a dose of about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1575 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200, or about 2240 mg.
32) The method of any one of embodiments 1-31, wherein the bispecific anti-EGFR/c-Met antibody is administered at a dose of 1050 mg.
33) The method of any one of embodiments 1-31, wherein the bispecific anti-EGFR/c-Met antibody is administered at a dose of 1400 mg.
34) The method of any one of embodiments 1-31, wherein the bispecific anti-EGFR/c-Met antibody is administered at a dose of 1575 mg.
35) The method of any one of embodiments 1-31, wherein the bispecific anti-EGFR/c-Met antibody is administered at a dose of 1600 mg.
36) The method of any one of embodiments 1-31, wherein the bispecific anti-EGFR/c-Met antibody is administered at a dose of 2100 mg.
37) The method of any one of embodiments 1-31, wherein the bispecific anti-EGFR/c-Met antibody is administered at a dose of 2240 mg.
38) The method of any one of embodiments 1-37, wherein the bispecific anti-EGFR/c-Met antibody is administered twice a week, once a week, once in two weeks, once in three weeks or once in four weeks.
39) The method of any one of embodiments 1-38 wherein the bispecific anti-EGFR/c-Met antibody is administered intravenously.
40) The method of any one of embodiments 1-38 wherein the bispecific anti-EGFR/c-Met antibody is administered subcutaneously.
41) The method of any one of embodiments 1-5, wherein the method further comprises determining levels of amphiregulin , and administering or providing for administration the bispecific anti-EGFR/c-Met antibody to the subject determined to have the EGFR lacking activating mutations and determined to be positive for amphiregulin.

The present invention will now be described with reference to the following specific, non-limiting examples.

### EXAMPLE 1

### Characterization of Non-Small Cell Lung Cancer (NSCLC) Patient-Derived Xenograft (PDX) Tumors Expressing EGFR which Lacks Activating Mutations

EGFR and MET protein levels, as determined by immunohistochemistry (IHC); signaling, as determined by proximity ligation assays (PLA); as well as tumor associated macrophage (TAM) content, were assessed in 39 NSCLC patient-derived xenograft (PDX) models having EGFR lacking activating mutations. The lack of activating mutations in EGFR was determined by whole exome sequencing (WES). The 39 NSCLC PDX tumors having EGFR lacking activating mutations, formalin fixed and paraffin embedded (FFPE), were obtained from Charles River Laboratory (CRL, Freiburg, Germany). The tumors included 19 adenocarcinomas and 20 epidermoid NSCLCs. Using IHC and PLA, a correlation was evaluated between receptor protein levels and receptor signaling for both EGFR and MET in these models.

For IHC studies, tissue sections were processed as described in Smith et. al. 2015 (Annotation of human cancers with EGFR signaling-associated protein complexes using proximity ligation assays. Matthew A. Smith et. al. 2015, Science Signaling, 8(359):ra4). Briefly, the sections were rehydrated and antigens were retrieved. Nonspecific binding was blocked by incubation with 1.5% bovine serum albumin (BSA), and incubated overnight in BSA in 0.5% PBST using rabbit antibodies targeting EGFR (Ventana; Clone 5B7) or with MET clone D1C2 (Cell Signaling) and phospho-MET clone D26 (Cell Signaling) Slides were washed twice with PBST, incubated with EnVision+anti-rabbit (K400311-2, Agilent) for 1 hour, and visualized by DAB (diaminobenzidine). Slides were counterstained with hematoxylin, rehydrated, and hard-mounted. To calculate H-score, staining intensity of cells was scored (0, 1+, 2+, 3+) and a percentage of cells at each intensity determined. The formula [1×(% cells 1+)+2×(% cells 2+)+3×(% cells 3+)] was then used to calculate H-scores ranging from 0 to 300 for each PDX model.

The Proximity Ligation Assay (PLA) was performed according to the published protocol (Annotation of human cancers with EGFR signaling-associated protein complexes using proximity ligation assays. Matthew A. Smith et. al. 2015, Science Signaling, 8(359):ra4). Briefly, slides containing 5-μm sections of FFPE PDX tumors were rehydrated through xylene and graded alcohols. Heat-induced epitope retrieval was carried out in tris-EDTA (pH 9) in a pressure cooker for 20 min and then cooled for 20 min. Nonspecific binding was blocked by incubation with 1.5% bovine serum albumin (BSA) at room temperature for 30 min. Primary antibodies were incubated overnight in 1.5% BSA in 0.5% phosphate-buffered saline (PBS)-Tween 20 (PBST) using rabbit antibody targeting EGFR (clone D38B1, Cell Signaling Technology) or ET clone D1C2 (Cell Signaling) diluted 1:300 and mouse antibody targeting Growth-factor Receptor-Bound Protein 2 (GRB2) (clone 81, BD Biosciences). PLA probes were rabbit (−) and mouse (+) and were detected with Duolink™ In Situ PLA Far Red kit (Sigma-Aldrich). Alexa Fluor 488-conjugated anti-cytokeratin was used to demarcate epithelial regions (clone AE1/AE3, eBioscience).

Confocal images were acquired on a Leica TCS SP5 AOBS (Acousto Optical Bream Splitter) laser scanning confocal microscope through a 40× 1.25 NA (numerical aperture) Plan Apochromat oil immersion objective lens (Leica Microsystems CMS GmbH). Diode (405) and HeNe (647) laser lines were applied to excite the samples, and tunable emissions were used to minimize crosstalk between fluorochromes. Z-stack (0.5-μm-thick slices) images for each sample were captured with photomultiplier detectors, and maximum projections were prepared with the LAS AF software version 2.6 (Leica Microsystems). Additional fluorescent images were acquired on a fully automated, upright Zeiss Axio-ImagerZ.1 microscope with a 40× 1.25 NA oil immersion objective, and DAPI and Cy5 filter cubes. Images were produced using the AxioCam MRm CCD (charge-coupled device) camera and Axiovision version 4.6 software suite (Carl Zeiss Inc.). All tissue-based PLA and AQUA analysis images were acquired using a 20× objective lens (dry) on an AQUA workstation (PM-2000, HistoRx) equipped with a fully motorized stage and DAPI, Cy3, FITC (fluorescein isothiocyanate), and Cy5 filter cubes. Images were saved as individual channels and exported as merged RGB TIFF images (Annotation of human cancers with EGFR signaling-associated protein complexes using proximity ligation assays. Matthew A. Smith et. al. 2015, Science Signaling, 8(359):ra4; MET-GRB2 Signaling-Associated Complexes Correlate with Oncogenic MET Signaling and Sensitivity to MET Kinase Inhibitors. Matthew A. Smith et. al. 2017, Clin Cancer Res. 2017, 23(22): 7084-7096).

The PLA scores were determined as previously described (Smith et. al. 2015). Briefly, PLA was manually quantified using a scoring criteria based on foci per cell (0, nondetectable; 1+, 1 to 5 foci per cell; 2+, >5 to 20 foci per cell; 3+, >20 foci per cell) and annotated as “high” (2+ to 3+in both cores) or “low” (0 to 1+).

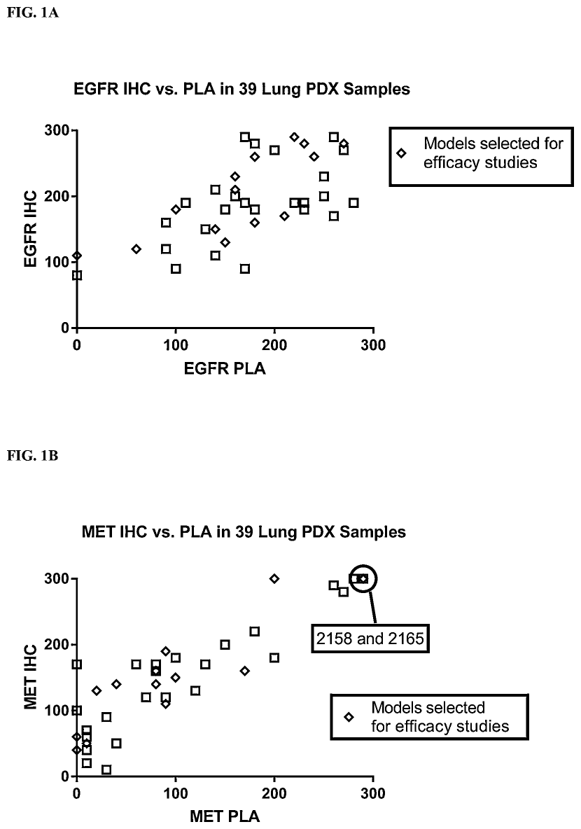

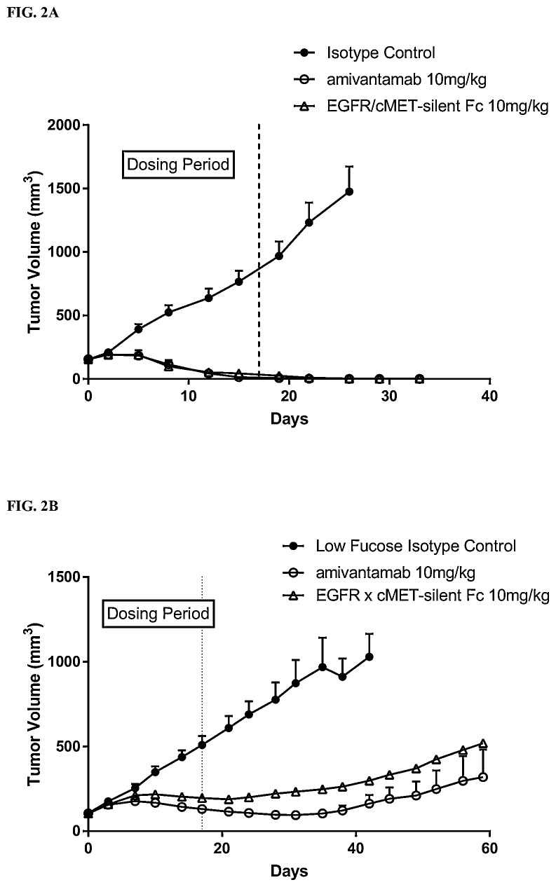

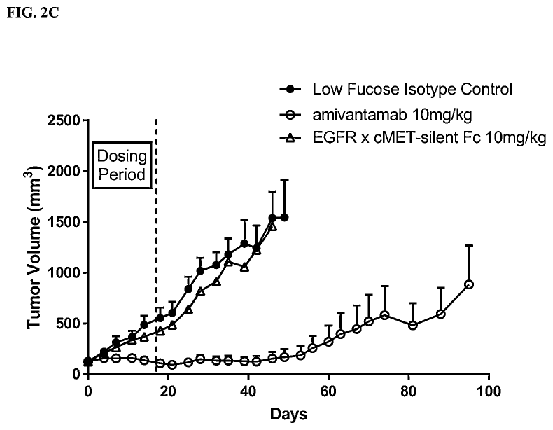

The results suggested that statistically significant correlations between the IHC and PLA scores were observed for both EGFR (r=0.63, p<0.0001, see FIG. 1A) and MET (r=0.83, p<0.0001, see FIG. 2A). Spearman's correlation was used to calculate r values, and two-tailed test was used to calculate p values (Prism Graphpad v.7.0.0). Specifically, p values for Spearman correlation coefficient were calculated using a permutation test if there were less than 17 XY pairs. Otherwise, the statistic

$$r \sqrt{\frac{n - 2}{1 - {r}^{2}}}$$

was compared to the Student's t-distribution with n−2 degrees of freedom where r is Spearman's correlation coefficient and n is the number of XY pairs.

### EXAMPLE 2

### Engagement of Fc Receptors is Not Required for Amivantamab Efficacy in NSCLC Models Having EGFR Lacking Activating Mutations

Next, the efficacy of amivantamab in NSCLC models having EGFR lacking activating mutations, was tested in vivo.

The in vivo studies were performed at Charles River Laboratory (CRL, Freiburg, Germany) in accordance with Janssen Animal and Care and Use Committee policies and procedures. Fourteen NSCLC PDX tumors were implanted subcutaneously in the flank of NMRI nu/nu mice (CRL) and were randomized into treatment groups when tumors reached 50-200 mm³. Treatments were administered twice weekly for 3 weeks by intraperitoneal injection of 10 mg/kg of either isotype control, or amivantamab, or an EGFR/MET bi-specific antibody having a silent Fc. The EGFR/MET-silent Fc antibody retains the EGFR and MET arms of amivantamab and has substitutions V234A/G237A/P238S/H268AN309L/A330S/P331S made to the heavy chains to statistically significantly decrease the affinity to Fcγ receptors and Cq1 complement. Tumors were measured by calipers and tumor volumes were calculated using the formula:(length×(width))/2. Percent tumor growth inhibition (% TGI) values were calculated at 7 days post-last dose using the formula: {1−[(Treatedₜ−Treatedᵢ)/(Controlₜ−Controlᵢ)]}×100.

The results suggested that amivantamab inhibited tumor growth in 13 of 14 PDX models tested (representative efficacy plots are shown in FIGS. 2A and 2B). Of these 13 models in which amivantamab displayed activity, the EGFR/MET-silent Fc antibody which lacks binding to Fc-receptors, was equipotent (FIGS. 2A and 2B). LXFA2158, a model with MET gene amplification, was the only PDX model in which the Fc-binding of amivantamab was critical to efficacy (FIG. 2C). This is consistent with the previously reported data (Smruthi Vijayaraghavan et. al. 2020, Molecular Cancer Therapeutics, 19(10):2044-2056) which demonstrated the necessity of the amivantamab Fc-interactions in MET amplified and EGFR mutant model systems.

### EXAMPLE 3

### Relationship Between Receptor Expression and Signaling to Amivantamab In Vivo Efficacy

Next, the relationship between receptor expression or signaling with amivantamab in vivo efficacy was evaluated.

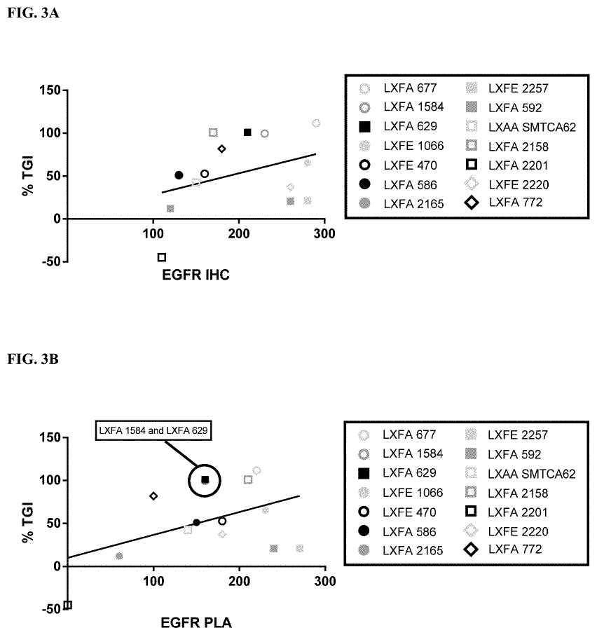

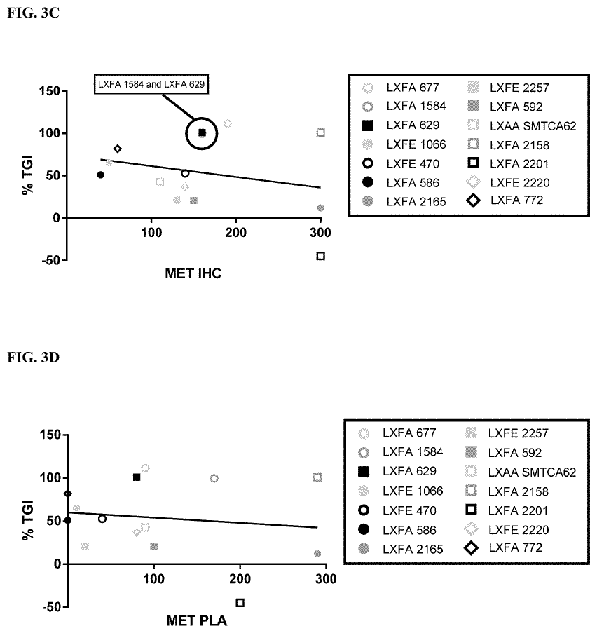

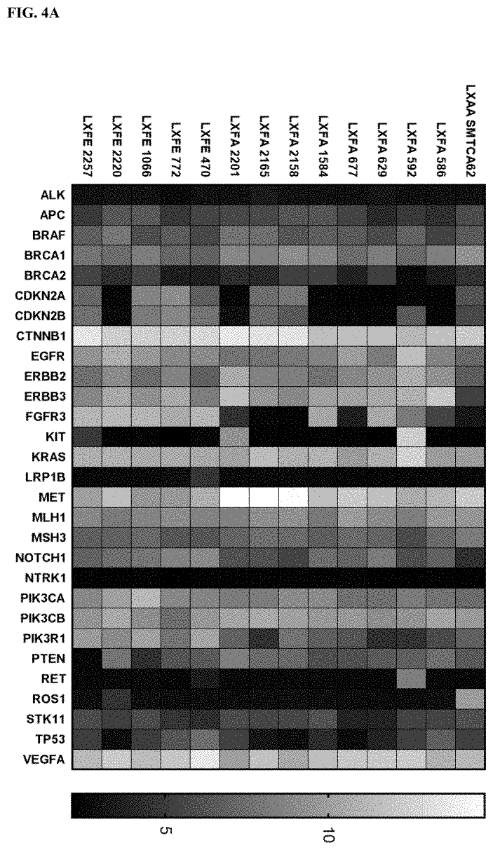

IHC and PLA assays were performed as described in Example 1. Amivantamab efficacy (% TGI) in PDX tumors having EGFR lacking activating mutations, was plotted in relation to the EGFR and MET IHC H-scores and PLA scores (see FIGS. 3A-3D). While positive correlations were determined between efficacy and both IHC and PLA for EGFR (IHC: r=0.397; PLA: r=0.22), neither correlation was statistically significant (p>0.05). No positive correlation between tumor efficacy and MET expression or signaling was observed.

### EXAMPLE 4

### Genomic Analysis of PDX Tumors

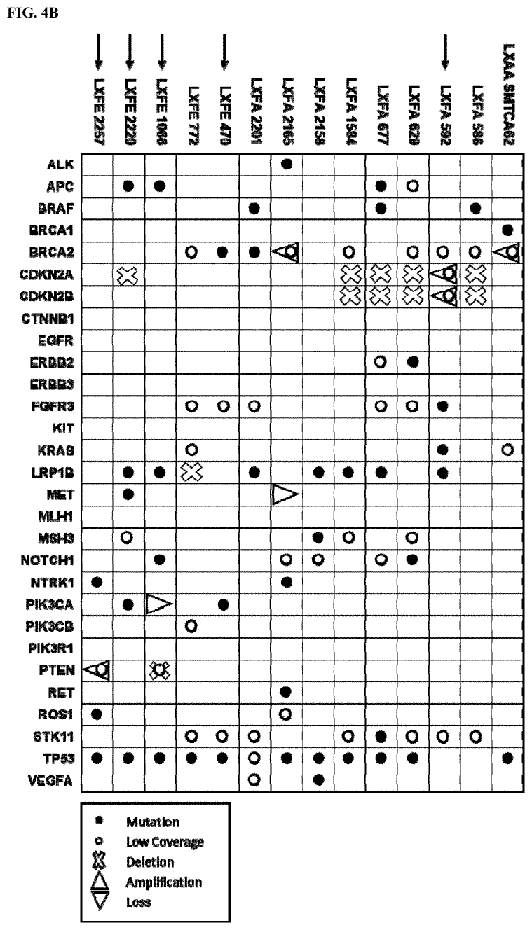

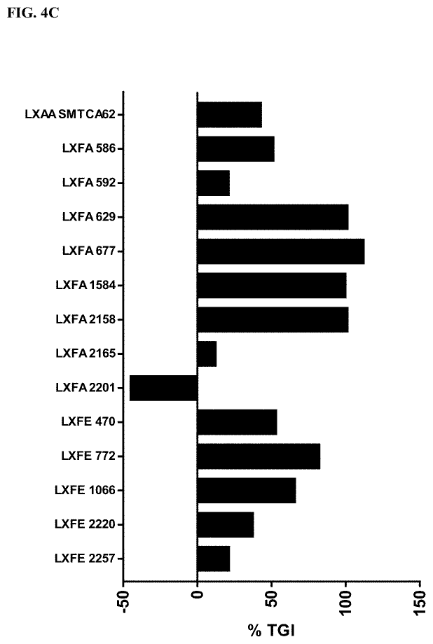

Next, the association of the expression and mutational status of the PDX tumors having EGFR lacking activating mutations, with the % tumor growth inhibition obtained in the Example 2 was evaluated. The expression data and mutational status of common oncogenes in the PDX tumors having EGFR lacking activating mutations, used in the Example 2, were provided and are fully owned by the Charles River Laboratory (Freiburg, Germany). The expression of the common oncogenes listed in FIG. 4A was determined by RNA-Seq, and the mutational status was determined by whole exome sequencing (FIG. 4B). While amivantamab was highly efficacious in many of these NSCLC PDX models having EGFR lacking activating mutations (FIG. 4C), mutations in the KRAS and PI3K pathways were identified as biomarkers indicative of decreased anti-tumor activity (see FIGS. 4A-4C).

When assessing the EGFR correlations to efficacy, we observed a subset of models in which the % TGI observed trended below the linear fit of the dataset, however the EGFR levels were relatively high (FIG. 3A). Interestingly, this subset of models was found to contain mutations in the KRAS or PI3K pathways and suggested that aberrant signaling in these pathways could reduce the activity of amivantamab.

### EXAMPLE 5

### PDX Tumors Lacking the KRAS and PI3K Pathway Mutations were Associated with Increased Amivantamab Efficacy

Next, the association of amivantamab efficacy (% TGI) and the sum of H-score and PLA score (see Table 1) was evaluated in a subgroup of PDX tumors from Example 2, wherein the tumors positive for the known oncogenic driver mutations downstream of EGFR and MET, such as KRAS and PI3K, were removed, see Table 2. The correlation coefficient and the p-value were calculated using Spearman's correlation and two-tailed test (Prism Graphpad 7.00), as described in the Example 1.

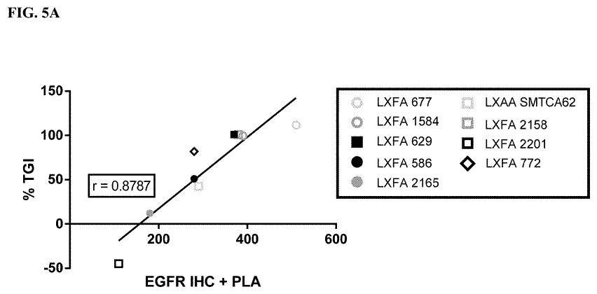

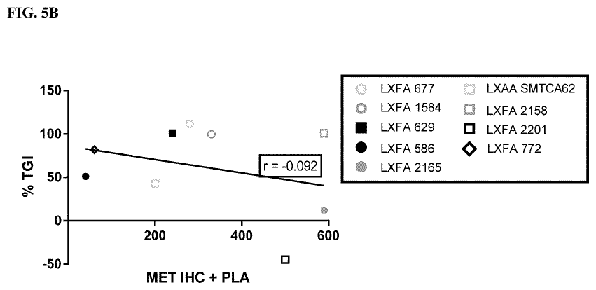

The results indicated that exclusion of tumors which harbored KRAS or PI3K pathway mutations resulted in a statistically significant correlation between amivantamab efficacy and EGFR expression and signaling (IHC+PLA−r=0.88, p=0.0032, see FIG. 5A). Indeed, models with EGFR H-score≥170 and no alternative driver mutations achieved prolonged tumor stasis or complete tumor regressions with amivantamab treatment (representative tumor growth curves are shown in FIGS. 2A and 2B). No correlation of amivantamab efficacy with MET expression and signaling was observed (FIG. 5B).

TABLE 1
IHC score (H-score), PLA-score, their sum, and the %
TGI, determined for PDX tumors having EGFR lacking
activating mutations, when treated with amivantamb.

| | EGFR | | | MET | | | |
|---|---|---|---|---|---|---|---|
| | H- | PLA | | H- | PLA | | |
| Model | Score | Score | Total | Score | Score | Total | % TGI |
| LXAA SMTCA62 | 150 | 140 | 290 | 110 | 90 | 200 | 42.63 |
| LXFA 1584 | 230 | 160 | 390 | 160 | 170 | 330 | 99.58 |
| LXFA 2158 | 170 | 210 | 380 | 300 | 290 | 590 | 100.95 |
| LXFA 2165 | 120 | 60 | 180 | 300 | 290 | 590 | 12.02 |
| LXFA 586 | 130 | 150 | 280 | 40 | 0 | 40 | 51.05 |
| LXFA 592 | 260 | 240 | 500 | 150 | 100 | 250 | 20.78 |
| LXFE 1066 | 280 | 230 | 510 | 50 | 10 | 60 | 65.47 |
| LXFE 2257 | 280 | 270 | 550 | 130 | 20 | 150 | 21.11 |
| LXFE 470 | 160 | 180 | 340 | 140 | 40 | 180 | 52.8 |
| LXFA 629 | 210 | 160 | 370 | 160 | 80 | 240 | 101 |
| LXFA 677 | 290 | 220 | 510 | 190 | 90 | 280 | 111.8 |
| LXFA 2201 | 110 | 0 | 110 | 300 | 200 | 500 | −44.74 |
| LXFE 2220 | 260 | 180 | 440 | 140 | 80 | 220 | 37.2 |
| LXFE 772 | 180 | 100 | 280 | 60 | 0 | 60 | 81.9 |

TABLE 2
KRAS and PI3K pathway mutations detected in PDX tumors.

| PDX Tumor model | KRAS | PIK3CA | PTEN |
|---|---|---|---|
| LXFA 592 | G12C | — | — |
| LXFE 470 | — | E545K | — |
| LXFE 2220 | — | H1047L | — |
| LXFE 1066 | — | amplification | deletion (associated with loss |
| | | | of expression) |
| LXFE 2257 | — | — | loss (associated with loss of |
| | | | expression) |

### EXAMPLE 6

### Analysis of the Association of EGFR Ligands' Expression and Amivantamab Efficacy in PDX Tumors Having EGFR Lacking Activating Mutations

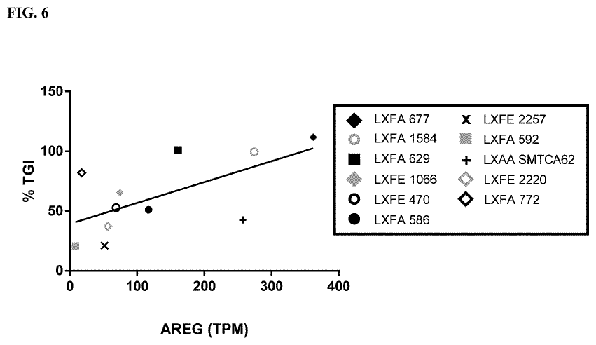

Next, the association of the expression of the EGFR ligands in the PDX tumors having EGFR lacking activating mutations, with the % tumor growth inhibition obtained in the Example 2 was evaluated. The expression of Epidermal growth factor (EGF), amphiregulin (AREG), transforming growth factor α (TGFα), heparin-binding EGF-like growth factor (HBEGF), betacellulin (BTC), epiregulin (EREG), and epigen (EPGN) in the PDX tumors having EGFR lacking activating mutations, used in the Example 2, was determined using RNA-Seq. These data were provided and are fully owned by the Charles River Laboratory (Freiburg, Germany). Amphiregulin expression was only available for 11 of the 14 NSCLC models in which in vivo efficacy was tested (data not available for LXFA 2158, LXFA 2165, and LXFA 2201). A statistically significant correlation (r=0.66; p=0.03) was found between amphiregulin expression and amivantamab in vivo efficacy (FIG. 6). This association between amphiregulin expression and amivantamab efficacy was independent of KRAS and PI3K pathway mutations. Therefore, amphiregulin expression may identify patients likely to gain clinical benefit from amivantamab treatments.

We claim:

1) A method of treating a subject having a cancer that is positive for EGFR and lacks an at least one EGFR-activating mutation, comprising administering a therapeutically effective amount of an isolated bispecific anti-epidermal growth factor receptor (EGFR)/hepatocyte growth factor receptor (c-Met) antibody to the subject having cancer that is positive for EGFR and lacks an at least one EGFR-activating mutation.

2) The method of claim 1, comprising:
a) providing a biological sample from the subject;
b) determining presence or absence of an EGFR-activating mutation in the sample;
c) administering or providing for administration the bispecific anti-EGFR/c-Met antibody to the subject determined to lack an EGFR-activating mutation.

3) The method of claim 1, wherein the at least one activating mutation is a mutation which increases at least one biological activity of EGFR.

4) The method of claim 3, wherein the at least one biological activity of EGFR is selected from the group consisting of tyrosine kinase activity, ligand-independent signaling, increased cell proliferation, signaling to MAPK/ERK pathways, gene transcription, dimerization (EGFR:EGFR), and heterodimerization (EGFR:HER2 or EGFR:HER3).

5) The method of claim 3, wherein the at least one activating mutation which increases at least one biological activity of EGFR comprise at least one mutation selected from the group consisting of L718Q, G719A, G719X (X being any amino acid), L861X (X being any amino acid), L858R, E746K, L747S, E749Q, A750P, A755V, V765M, C797S, L858P or T790M substitution, deletion of E746-A750, deletion of R748-P753, insertion of Ala (A) between M766 and A767, insertion of Ser, Val and Ala (SVA) between S768 and V769, insertion of Asn and Ser (NS) between P772 and H773, insertion of one or more amino acids between D761 and E762, A763 and Y764, Y764 and Y765, M766 and A767, A767 and V768, S768 and V769, V769 and D770, D770 and N771, N771 and P772, P772 and H773, H773 and V774, V774, C775, one or more deletions in EGFR exon 20, one or more insertions in EGFR exon 20, S768I, L861Q and G719X (X being any amino acid).

6) The method of claim 1, wherein the method further comprises determining presence or absence of at least one mutation in any one gene selected from the group consisting of KRAS, PIK3CA, and PTEN, and administering or providing for administration the bispecific anti-EGFR/c-Met antibody to the subject determined to have the EGFR lacking activating mutations and determined to lack at least one mutation in any one gene selected from the group consisting of KRAS, PIK3CA, and PTEN.

7) The method of claim 6 wherein the at least one mutation in KRAS is selected from the group consisting of G12V, G12C, G12A and G12D.

8) The method of claim 7 wherein the at least one mutation in KRAS is G12C.

9) The method of claim 8 wherein the at least one mutation in PI3K is selected from the group consisting of E545K, H1047L, and PI3K amplification.

10) The method of claim 6 wherein the at least one mutation in PTEN is PTEN deletion.

11) The method of claim 1, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

12) The method of claim 11, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

13) The method of claim 11, wherein the bispecific anti-EGFR/c-Met antibody is an IgG1 isotype.

14) The method of claim 11, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

15) The method of claim 11, wherein the bispecific anti-EGFR/c-Met antibody comprises one or more Fc silencing mutations.

16) The method of claim 11 wherein the one or more Fc silencing mutations decrease affinity to Fcγ receptors.

17) The method of claim 15 or 16 wherein the one or more Fc silencing mutations comprise V234A/G237A/P238S/H268A/V309L/A330S/P331S.

18) The method of claim 11, wherein the bispecific anti-EGFR/c-Met antibody comprises a biantennary glycan structure with a fucose content between about 1% to about 15%.

19) The method of claim 1, wherein the subject is relapsed or resistant to treatment with one or more prior anti-cancer therapies.

20) The method of claim 19, wherein the one or more prior anti-cancer therapies comprises one or more chemotherapeutic agents, checkpoint inhibitors, targeted anti-cancer therapies or kinase inhibitors, or any combination thereof.

21) The method of claim 20, wherein the one or more prior anti-cancer therapies comprises carboplatin, paclitaxel, gemcitabine, cisplatin, vinorelbine, docetaxel, palbociclib, crizotinib, PD-(L)1 axis inhibitor, an inhibitor of EGFR, an inhibitor of c-Met, an inhibitor of HER2, an inhibitor of HER3, an inhibitor of HER4, an inhibitor of VEGFR, an inhibitor of AXL, erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib, or any combination thereof.

22) The method of claim 1, wherein the subject is treatment naïve.

23) The method of claim 1, wherein cancer that is positive for the EGFR lacking activating mutations is positive for at least one mutation in a gene selected from the group consisting of ALK, APC, BRAF, BRCA1, BRCA2, CDKN2A, CDKN2B, CTNNB1, ERBB2, ERBB3, FGFR3, KIT, LRP1B, MET, MLH1, MSH3, NOTCH1, NTRK1, RET, ROS1, STK11, TP53, and VEGFA.

24) The method of claim 1, wherein the cancer is lung cancer, gastric cancer, colorectal cancer, brain cancer, cancer derived from epithelial cells, breast cancer, ovarian cancer, colorectal cancer, anal cancer, prostate cancer, kidney cancer, bladder cancer, head and neck cancer, pharynx cancer, cancer of the nose, pancreatic cancer, skin cancer, oral cancer, cancer of the tongue, esophageal cancer, vaginal cancer, cervical cancer, cancer of the spleen, testicular cancer, gastric cancer, cancer of the thymus, colon cancer, thyroid cancer, liver cancer, hepatocellular carcinoma (HCC) or sporadic or hereditary papillary renal cell carcinoma (PRCC), or any combination thereof.

25) The method of claim 24, wherein lung cancer is non-small cell lung cancer (NSCLC), small cell lung cancer (SCLC) or lung adenocarcinoma, pulmonary sarcomatoid carcinoma or any combination thereof.

26) The method of claim 1, comprising further administering one or more anti-cancer therapies to the subject.

27) The method of claim 26, wherein the one or more anti-cancer therapies comprises chemotherapy, radiation therapy, surgery, a targeted anti-cancer therapy, a kinase inhibitor, or any combination thereof.

28) The method of claim 20, wherein the kinase inhibitor is an inhibitor of EGFR, an inhibitor of c-Met, an inhibitor of HER2, an inhibitor of HER3, an inhibitor of HER4, an inhibitor of VEGFR or an inhibitor of AXL.

29) The method of claim 28, wherein the kinase inhibitor is erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib.

30) The method of claim 1, wherein the bispecific anti-EGFR/c-Met antibody is administered at a dose of between about 140 mg to about 2240 mg.

31) The method of claim 30, wherein the bispecific anti-EGFR/c-Met antibody is administered at a dose of about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1575 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200, or about 2240 mg.

32) The method of claim 30, wherein the bispecific anti-EGFR/c-Met antibody is administered at a dose of 1050 mg.

33) The method of claim 30, wherein the bispecific anti-EGFR/c-Met antibody is administered at a dose of 1400 mg.

34) The method of claim 30, wherein the bispecific anti-EGFR/c-Met antibody is administered at a dose of 1575 mg.

35) The method of claim 30, wherein the bispecific anti-EGFR/c-Met antibody is administered at a dose of 1600 mg.

36) The method of claim 30, wherein the bispecific anti-EGFR/c-Met antibody is administered at a dose of 2100 mg.

37) The method of claim 30, wherein the bispecific anti-EGFR/c-Met antibody is administered at a dose of 2240 mg.

38) The method of claim 1, wherein the bispecific anti-EGFR/c-Met antibody is administered twice a week, once a week, once in two weeks, once in three weeks or once in four weeks.

39) The method of claim 1, wherein the bispecific anti-EGFR/c-Met antibody is administered intravenously.

40) The method of claim 1, wherein the bispecific anti-EGFR/c-Met antibody is administered subcutaneously.
